(19) United States
(12) Patent Application Publication
(10) Pub. No.: US 2009/0125330 A1
(43) Pub. Date: May 14, 2009

(54) Non-invasive medical data collecting assembly

(76) Inventor: Eric Sebban, Hollywood, FL (US)

Correspondence Address: MALLOY & MALLOY, P.A.;Historic Coral Way, 2800 S.W. Third Avenue, Miami, FL, 33129, US

(21) Appl. No.: 11/983,613

(22) Filed: Nov. 9, 2007

(51) Int. Cl. G06Q 10/00; A61B 5/01; G06Q 50/00

(52) U.S. Cl. 705/3; 600/549; 705/2

(57) ABSTRACT

An assembly structured to collect medical data from at least one but more practically a plurality of patients, wherein the medical data collected includes, but is not limited to, temperature readings. A hand-held device or housing is disposable in an operative position in spaced relation to a predetermined target of the patient. A sensor assembly is operative to collect the intended medical data and a memory assembly serves to concurrently store the collected data. A display assembly is operatively associated with a control assembly wherein the collected medical data from any one of the plurality of patients may be selectively accessed, reviewed on the display assembly and be correlated with additional identification information corresponding to each of the plurality of patients from which the collected data originated. A docking station may be removably connected to the housing and includes a data transmitting assembly structured to access the medical data in the memory assembly and transmit it to a remote processor or like facility for further storage and/or processing.

### BACKGROUND OF THE INVENTION

1. Field of the Invention

The present invention is directed to a non-invasive medical data collecting assembly structured to collect temperature and/or other medical data utilizing a hand-held device operatively disposed relative to a patient. A memory assembly, a display assembly and a control assembly are all operatively interconnected and cooperatively structured to facilitate the storage of medical data from a plurality of different patients which may be selectively and repeatedly accessed, displayed and stored. Data transmitting capabilities are provided to transmit collected medical data from the memory assembly to a remote processing facility.

2. Description of the Related Art

The human body, as well as animals and other objects, whether solid, liquid or gas, release energy by radiation, wherein the strength of the radiation is indicative of the temperature of the object. As such, medical data such as the temperature of a human or animal patient can be acquired by a determination of the strength of the radiation emanating from the patient.

The method of temperature measurements by use of heat radiation possesses many advantages and overcomes certain disadvantages and problems associated with more conventional thermometers or like temperature acquiring devices. As such, the methods for measuring temperature can be classified into two categories including, contact and non-contact type devices. In the case of the contact-type of thermometer or temperature measuring device, heat is transferred directly to the temperature measuring instrument through a contact surface or interface by means of physically engaging the device or a specific portion thereof with a target area of the patient being examined. In contrast, a non-invasive thermometer or temperature acquiring assembly may be disposed a spaced distance from the target area of the individual and is structured to sense radiation as it emanates from the target area of the individual.

In many instances, the result of temperature measurement in a non-invasive manner, as set forth above, is more accurate as compared to the contact-type devices prevalent in previous years. This is due in part to the fact that the influence of the temperature sensing element on the target is smaller, particularly in the case when the heat capacity of the target is relatively small. In cases of medical examination, it may be troublesome, impractical or in certain instances even dangerous to utilize a contact-type of heat measuring device, based on the location or target area on the patient from which the temperature is to be taken. Accordingly, the development of thermometers and medical devices for the collecting of medical data including, but not limited to, temperatures has significantly advanced. As such it is now recognized that the non-invasive temperature acquiring devices may be preferable for many temperature acquiring applications.

Further, the use of infrared technology in acquiring temperatures by the non-invasive technique may also be preferred in many instances. In prior years, infrared thermometers frequently contemplated the insertion of a probe portion thereof into the ears or other appropriate orifices of the patient. However, advancement in infrared thermometers and the technology associated therewith allow for accurate readings of temperature. Even with such advancements in infrared thermometers, there are still certain limitations which are not available with conventional and/or commercially available devices.

Accordingly, there is a need in this area for an improved, proposed non-invasive medical data collecting assembly which is capable of storing temperatures or other medical data collected from a plurality of patients or from one patient successively over a period of time. As such, a new and proposed medical data collecting assembly should incorporate adequate memory capabilities for the storage of a significant number of collected temperatures or other medical data, as well as the ability to easily and quickly access the stored temperatures. Further, such a proposed and improved medical data collecting assembly should be capable of identifying each of the temperatures or like collected data with the individual patients and possibly include other required information such as time, date, etc. when the medical data was collected.

Finally, yet an additional feature of such an improved and proposed medical data collecting assembly should be the ability to download collected medical data to one or more additional, remotely located processing facilities. Such processing facilities may assume a wide variety of structural and operative features including a variety of computer facilities having significant storage capacity and the ability to communicate, on an interactive basis with the data collecting assembly as well as any one of a plurality of other remotely located processing facilities.

### SUMMARY OF THE INVENTION

This invention relates to a device structured to collect medical data from at least one patient but more practically a plurality of patients in a non-invasive manner. The medical data collected may include the temperature of the patient or patients but may also include additional and/or other information such as blood pressure, glucose levels, etc.

Moreover, the medical data collection assembly of the present invention includes a hand held device comprising a housing dimensioned and configured to be held and operated by a single hand of a user. When so used, the housing is disposed in a predetermined operative position relative to the patient being examined. As will be more specifically described hereinafter, the operative position comprises the hand-held housing being preferably disposed a predetermined spaced distance from a target area of the patient such as, but not limited to, the patient's forehead. Therefore, the hand-held housing of the device includes a plurality of interconnected or otherwise operatively associated components which cooperate to collect the predetermined medical data and store a plurality of readings representative of the data collected from one or a plurality of patients in a readily accessible manner.

In addition, a display assembly is mounted on the hand-held device and is interconnected or operatively associated to communicate with both a sensor assembly as well as a memory assembly. The sensor assembly is structured to determine and obtain the intended medical data and the memory assembly is structured to store the medical data, once obtained by the sensor assembly. A control assembly is disposed on the housing and is accessible and readily operable from an exterior thereof such as by the hand of a user serving to grip, support and position the housing relative to the patient. Therefore, the control assembly which may include a plurality of hand manipulated buttons, switches, etc. mounted on the exterior of the housing serve to activate, operate and/or accomplish interactive communication between the various components including, but not limited to the sensor assembly, memory assembly and display assembly.

Further, the control assembly, as well as the interactive communicative features of the sensor assembly and memory assembly in association with the display assembly, serves to activate the sensor assembly when the hand-held housing is in the aforementioned operative position in spaced, non-invasive relation to a patient. Further operation of the control assembly will serve to activate the sensor assembly facilitating the collection of the intended medical data. Substantially concurrently to such collection of data, the control assembly may be further operated to transfer the collected data directly to the display assembly. Alternatively the collected data may be automatically transferred and/or stored in the memory assembly. In either case, the display assembly is structured and interactively associated with both the sensor assembly and the memory assembly to display medical data which has been just collected from a patient or which has been stored and subsequently accessed from the memory assembly.

Moreover, the memory assembly is structured to store and permit selective access each of a predetermined number of collected data measurements such as, but not limited to, a plurality of temperatures obtained from a plurality of patients. In the alternative, a plurality of temperatures can be obtained from a single patient over a predetermined time and sequentially stored upon collection. Operation of the control assembly will serve to access any of the collected medical data stored in the memory assembly thereby facilitating observation or viewing thereof on the display assembly. Additional features of the control assembly being interactive with the memory assembly, is the entering of information data corresponding with each of the collected medical data inputs stored in the memory assembly. More specifically, “identification information” representative of the identity of each of the plurality of patients may be input by an appropriate user interface, preferably on the hand held device. As such, each medical data input, such as temperature, obtained from one or more patients may be directly associated with appropriate identification information such that the collected data may be readily identified and directly associated with a given patient. It is also to be noted that the user interface may enter additional and/or supplementary information, such as time of taking the temperature or other medical data collected as well as other appropriate information which may be used to more accurately evaluate the temperature or other medical data collected.

Another operative and structural feature of at least one preferred embodiment of the present invention is the inclusion of a docking station which is removably connected and/or disposed in supporting relation to the hand-held housing portion of the collecting assembly. In addition, the docking assembly may include a data transmitting assembly and be otherwise structured to be connected or disposed in communicating relation with the memory assembly of the hand-held housing. More specifically, when the housing is removably supported in an operative orientation on the docking assembly, there is an interactive communication between the data transmitting assembly associated with the docking assembly and the memory assembly associated with the hand-held housing. Such operative, interactive communication serves to effectively download the stored, collected medical data into the data transmitting assembly and/or any supplementary storage capability associated therewith. Alternatively, instead of downloading of the collected and stored medical data from the memory assembly into the data transmitting assembly, one embodiment of the present invention comprises the direct access and transmission of the stored data in the memory assembly by the data transmitting assembly to a remote processing facility. The interactive communication between the data transmitting assembly and the memory assembly may be accomplished by a hard wire connection, wireless connection, including the utilization of BlueTooth protocol or by other appropriate communication means.

Once the collected medical data is accessed or downloaded from the memory assembly of the hand-held housing, the data transmitting assembly is further structured to communicate the collected medical data to a remote site for storage and/or further processing. As such, the data transmitting assembly may be structured for one way or two way communication with a remote processing facility such as, but not limited to, a remote computer. The remote processing facility may therefore be capable of additional, larger capacity storage, selective accessing and review, further transmission of the received, collected medical data to additional remote processing facilities and/or other processing procedures.

At the same time it is emphasized that the memory assembly associated with the hand-held housing portion of the collecting assembly of the present invention may still maintain the plurality of collected medical data stored therein after being accessed by the data collecting assembly. Accordingly, the collected medical data may be individually accessed and displayed on the aforementioned display assembly even after transmission of such medical data by the data transmitting assembly has taken place.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a rear perspective view of the medical data collecting assembly of the present invention.

FIG. 2 is a front perspective view of the embodiment of FIG. 1.

FIG. 3 is a front view of the embodiment of FIGS. 1 and 2.

FIG. 4 is a rear view of the embodiment of FIGS. 1-3.

FIG. 5 is a schematic representation of various operative components associated primarily, but not exclusively, with the hand-held housing portion of the assembly of the embodiments of FIGS. 1-4.

FIG. 6 is a schematic view of the various operative components associated with a docking assembly which is part of the embodiments of FIGS. 1-4.

Like reference numerals refer to like parts throughout the several views of the drawings.

### DETAILED DESCRIPTION OF THE PREFERRED EMBODIMENT

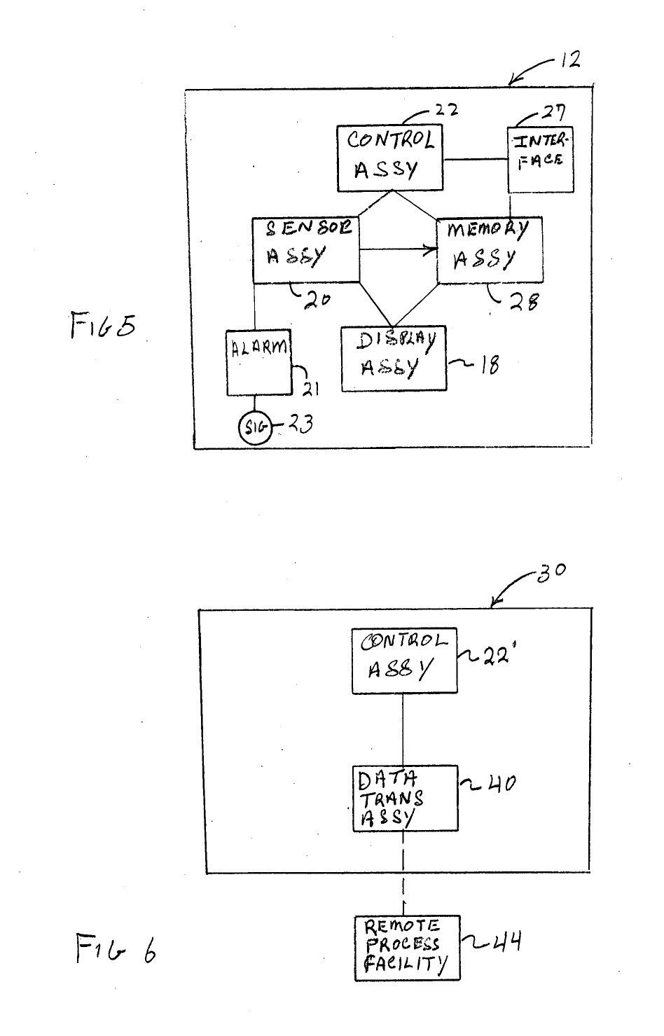

As represented in the accompanying drawings, the present invention is directed to a non-invasive medical data collecting assembly generally indicated as 10. Moreover, the collecting assembly 10 includes a hand held device having a housing generally indicated as 12 structured to be hand operated during the data collecting procedure. Structural details of the housing 12 include a handle portion generally indicated as 14 and a head or containment portion generally indicated as 16 on which a display assembly 18 and a sensor assembly 20 are disposed. As should be apparent, the operative components, circuitry, etc. of both the display assembly 18 and the sensor assembly 20 are contained within an at least partially hollow interior of the head portion 16 of the device or housing 12. As such these components are interconnected or otherwise operatively associated so as to establish interactive communication therebetween as will be explained in greater detail with regard to the schematic representation of FIG. 5.

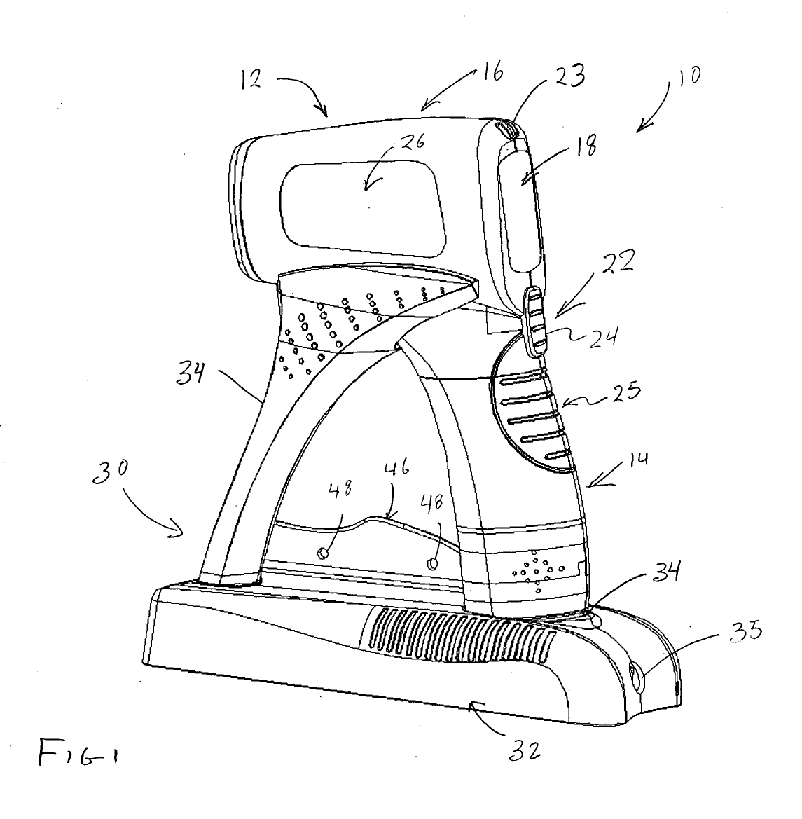

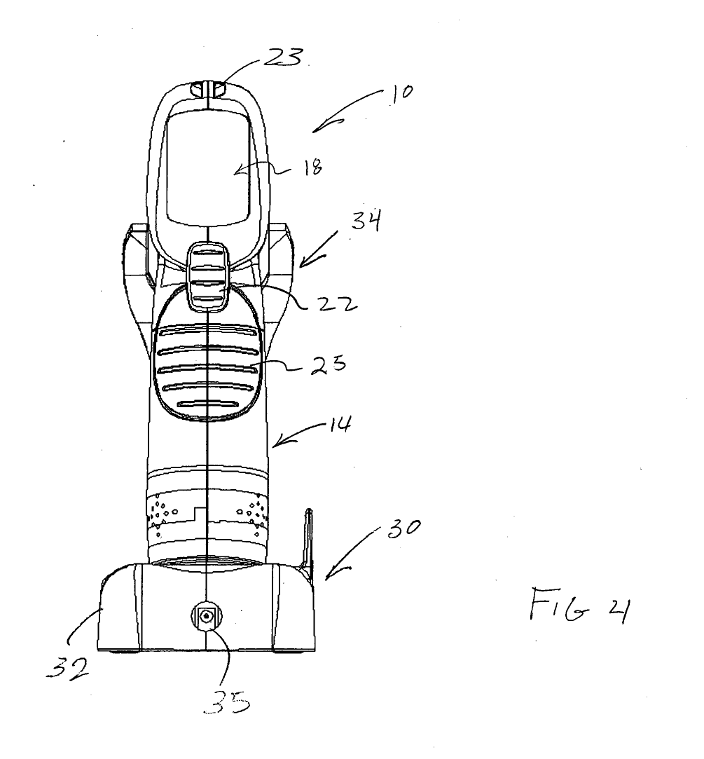

A control assembly is generally referred to, at least in part, as 22 and includes one or a plurality of control buttons or other control structures 24, 24, etc, which may be manipulated by the hand and/or fingers of the user which serve to grip and support the housing 12. A plurality of the control buttons 24, 25, etc, are represented in FIGS. 1 and 4. However, a number of other operative buttons or control members may also define at least a portion of the control assembly 22 and may be located on the exterior of the housing 12 in a readily accessible position on the housing 12. By way of example, such additional buttons or control members may be located on a panel area generally indicated as 26. Such additional control buttons or like structures, while not shown for purposes of clarity, may vary in number, size, configuration and structure to facilitate effective operation of the device or housing 12 of the assembly 10.

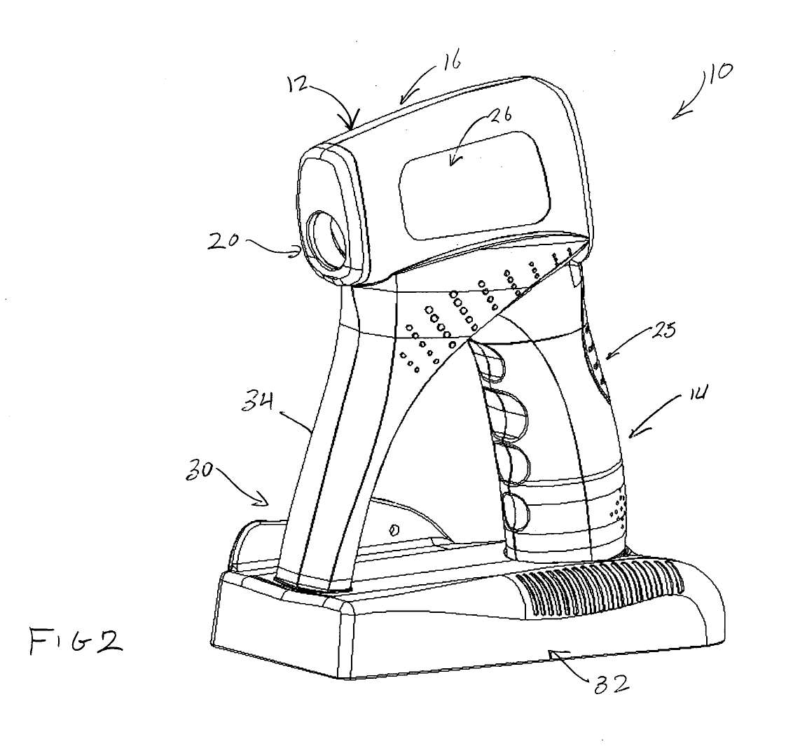

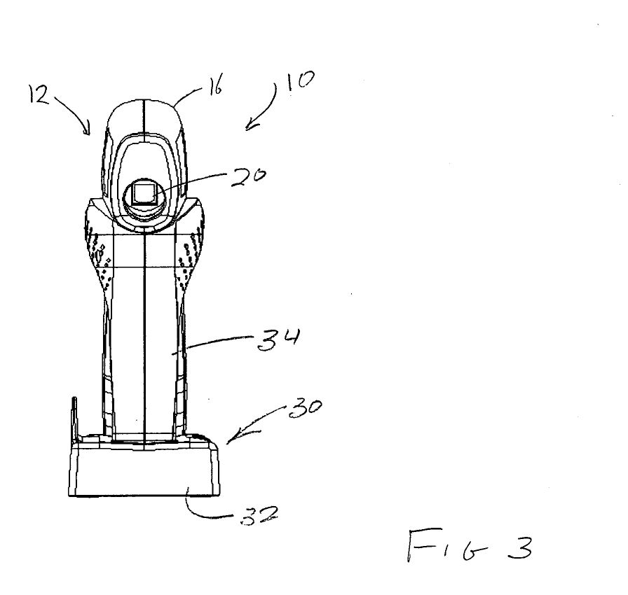

Another feature of the data collecting assembly 10 includes a docking station generally indicated as 30. The docking station 30 preferably includes a base portion 32 and a supporting yoke, pedestal or like member 34 which serves to support and facilitate engagement with the device or housing 12 in the manner represented in FIGS. 1-4. In addition, the base 32 includes an input or receiving terminal 34 which may be structured to establish operative, interactive communication between a data transmitting assembly, generally indicated as 40 in FIG. 6, and one or more of the various operative components of the hand-held device or housing portion 12. Such operative components which are structured to establish interactive communication with the data transmitting assembly 40 include the control assembly 22, sensor assembly 20, memory assembly 28 and possibly the display assembly 18. As will also be explained in greater detail hereinafter with specific regard to the operational features and characteristics of the assembly 10, the memory assembly 28 is also included within the collecting assembly 10 and may be directly associated with the hand-held housing 12. Base 32 of the docking station 30 also includes an input socket or like junction port 35 for receipt of a power cord and/or communication line or both.

Accordingly, as represented in the various Figures, the hand-held housing 12 is removably supported and/or connected to the base 32 of the docking assembly 30. When in such a docked position or orientation the various components of the housing 12 are operatively associated by hardwire connection or wireless interface with the various operative components of the docking assembly 30. Further, the operative components of the hand-held housing 12 may be powered by a battery or battery pack which may be replaceable or which may be rechargeable, such as when the housing 12 is in its docked orientation as represented in FIGS. 1-4.

Additional structural and operative features will be explained with primary reference to FIGS. 5 and 6, schematically representing the interactive, operative association of the various components associated with both the hand-held device or housing portion 12 of the assembly 10 as well as the docking station 30 of the assembly 10. More specifically, the control assembly 22 is operable through hand manipulation of the operating or control buttons 24, 25, etc. on the housing 12. When manipulated these control buttons serve to activate and/or operate the operative components at least associated with the housing 12. As set forth above, the operative components associated with the housing 12 include the sensor assembly 20 which is preferably operable through the provision of infrared technology such as, but not limited to, the use of Heimann infrared technology. This infrared technology is especially conceived to measure the bodies' temperature instantly and is adaptable for the obtaining or collection of other medical data including glucose levels, blood pressure, etc.

As such, the control assembly is operatively associated with or connected to the sensor assembly 20 as well as the memory assembly 28. The memory assembly 28 is capable of storing collected medical data such as, but not limited to, a predetermined number or plurality of temperature readings from one and/or a plurality of patients. As is schematically represented in FIG. 5, the sensor assembly 20 is linked for interactive communication with the memory assembly 28. Therefore, the determination or collection of the intended medical data or temperature, when achieved, is transferred to the memory assembly 28 for storage. Further, the display assembly 18 is interconnected to both the sensor assembly 20 and the memory assembly 28 and may be activated either independently or automatically by hand manipulation of the control assembly 22. More specifically, upon the hand-held housing 12 being disposed in a preferred or predetermined operative position, in spaced relation to a target area of a patient such as the forehead, the intended medical data or temperature will be collected through operation of the sensor assembly. Such collected medical data will be transferred to memory assembly and be concurrently displayed for observation on the display assembly 18.

The user of the hand-held housing 12 may eventually pass on to a plurality of other patients and sequentially obtain the intended medical data, such as the temperatures, therefrom. Each collected temperature reading or other intended medical data is successively stored for independent access in the memory assembly 28. Other manipulation or operation of the control assembly 22 may serve to access the memory assembly 28 and in particular call for any of the plurality of previously collected temperature readings or other medical data by direct access or by incorporating a scrolling feature in the control assembly 22. Any accessed medical data which has been collected can then be displayed on the display assembly 18 for observation.

Further, the memory assembly 28 may be directly associated with a user interface 27 such as a plurality of input buttons which may be mounted at a convenient location on the hand-held housing 12 such as on the panel 26. Such interface 27 allows the input of identifying information so as to correspond each temperature reading or other collected medical data with a specific patient. Such identifying information may also involve other supplementary information such as the time or date when the temperature or other medical data was collected. Accordingly, proper operation of the control assembly through hand manipulation of the various control structures 24, 25, etc. on the hand-held housing 12 will serve to access any of the previously collected temperature readings or medical data as well as the identifying information associated therewith.

Other features associated with the hand-held device or housing 12 include an alarm assembly generally indicated as 21 which may be automatically activated upon the sensor assembly 20 determining that the temperature of the patient is outside of acceptable parameters. More specifically, when the sensor assembly 20 determines a temperature of, by way of example only, 101° F. or more, the alarm assembly 21 may be automatically activated. The activation of the alarm assembly 21 may be evidenced by an appropriate icon or other display on the display assembly 18 or alternatively by a flashing light or audio indicator 23 appropriately positioned on the hand-held housing 12.

As set forth above, the medical data collecting assembly 10 also includes a docking station 30 as represented in FIGS. 1-4 and as schematically represented in FIG. 6. The docking assembly 30 is removably connected to and serves to support the hand-held housing 12 such as by additional support by the upwardly protruding yoke or pedestal 34. The opposite or free end of the handle 14 is then removably inserted or disposed within the inlet or receiving socket 34 so as to facilitate data transfer between the memory assembly 28 and a data transmitting assembly 40, the latter being operatively associated with the docking station 30.

For purposes of clarity, a control assembly in FIG. 6 is represented as 22′ and may be associated with the control assembly 22 operatively associated with the hand-held device 12. However, when the housing 12 is connected to the docking station 30, the control assembly 22 and the control assembly 22′ may in fact represent the same structure and operative function so as to facilitate data transfer from the memory assembly 28 to the data transmitting assembly 40, such as by manipulation of the control members 24, 25, etc. It is again emphasized that linking communication may be accomplished by a hardwire connection or wireless and/or BlueTooth capabilities between the hand-held housing 12 and the docking station 30 and more specifically the memory 28 and the data transmitting assembly 40.

The data transmitting assembly 40 may be structured to include either one way or two way communication and as such may be in the form of a wireless and/or hardwire transmitter or transceiver. In either of these structural and operative modifications, the medical data collected and stored within the memory assembly 28 may be transferred in whole or in part to the data transmitting assembly 40 through proper operation and activation of the control assembly 22, 22′. However, the interactive communication between the memory assembly 28 and the data transmitting assembly 40 is such that the collected data stored in the memory assembly 28 may be efficiently downloaded to the data transmitting assembly 40 or transmitted directly to a remote processing facility 44 through the data transmitting assembly 40. At the same time all medical data which has been collected and stored in the memory assembly 28 may remain there to facilitate quick and efficient access by a user of the hand held device 12 even after the collected medical data has been transmitted by the data transmitting assembly 40.

The data transmitting assembly 40 through appropriate wired or wireless transmission is capable of transmitting, transferring or uploading the collected medical data from the memory assembly 28 to the remote processing facility 44. Further, the remote processing facility 44 may be located in the same generally vicinity as the medical data collecting assembly 10 or alternatively may be located a significant, remote distance therefrom. Further, the remote processing facility 44 may be in the form of any one of a plurality of different computer facilities capable of permanent and/or larger capacity storage. Moreover, the remote processing facility 44 may be capable of further communication with other processing assemblies so as to distribute the collected medical data in a timely and efficient manner for further observation and evaluation.

Yet other features associated with the medical data collecting assembly 10 as represented in FIGS. 1-4 is a mounting structure 46 having through holes 48. Holes 48 may be dimensioned, disposed and configured to receive any type of appropriate connector for mounting the docking assembly 30 on an appropriate wall or like supporting surface. Alternatively, the under surface portion of the base 32 of the docking station 30 may be mounted on a horizontal support surface if preferred

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. An assembly structured to collect medical data from at least one individual, said assembly comprising:
a housing disposable in an operative position relative to the patient,
a sensor assembly disposed on said housing and structured to non-invasively determine the medical data while said housing is in said operative position,
said operative position comprising said housing disposed within a predetermined spaced distance from the patient,
a memory assembly cooperatively structured with said sensor assembly to concurrently store collected medical data of a plurality of patients,
a display assembly disposed on said housing and structured to view collected medical data, and
a control assembly at least partially disposed on said housing and structured to selectively determine the medical data maintained in said memory facility.

2. An assembly as recited in claim 1 further comprising a medical data transmitting assembly cooperatively structured with said memory assembly to communicate the medical data maintained in said storage assembly to a remote processing facility.

3. An assembly as recited in claim 2 further comprising a docking station operatively and removably connected to said housing.

4. An assembly as recited in claim 3 wherein said data transmitting assembly is at least partially disposed on said docking station.

5. An assembly as recited in claim 2 wherein said data transmitting assembly is further structured for wireless communication between said memory assembly and the remote processing facility.

6. An assembly as recited in claim 5 further comprising a docking station operatively and removably connected to said housing; said data transmitting assembly at least partially disposed on said docking station.

7. An assembly as recited in claim 1 wherein said sensor assembly comprises infrared capabilities and is structured to determine medical data at least partially defined by a temperature of the patient.

8. An assembly as recited in claim 7 wherein said operative position comprises said housing and said sensor assembly disposed a predetermined spaced distance from a target area on the patient.

9. An assembly as recited in claim 8 wherein said sensor assembly is structured to determine a temperature of the patient when disposed in said operative position, said operative position defined by a predetermined spaced distance from the target area defined by the forehead of the patient.

10. An assembly as recited in claim 1 further comprising an alarm assembly operatively associated with at least said sensor assembly and structured to generate an alarm upon the medical data exceeding predetermined parameters.

11. An assembly as recited in claim 1 wherein said control assembly is further structured to enter identification information and correlate the collected medical data with a corresponding one of the plurality of patients.

12. An assembly as recited in claim 11 wherein said control assembly comprises search capabilities operatively associated with said memory assembly and structured to facilitate selective access of collected medical data and identification information of any of the plurality of patients.

13. An assembly as recited in claim 12 further comprising a data transmitting assembly cooperatively structured with said memory assembly to communicate at least the medical data maintained in said storage assembly to a remote processing facility.

14. An assembly as recited in claim 13 wherein said data transmitting assembly is structured to communicate collected medical data and identification information of selected patients to the remote processing facility.

15. An assembly as recited in claim 14 wherein said memory assembly and said control assembly are cooperatively structured to maintain the medical data and the identification information in said memory assembly subsequent to transmission to the remote processing facility.
